(19) United States
(12) Patent Application Publication
(10) Pub. No.: US 2026/0083781 A1
(43) Pub. Date: Mar. 26, 2026

(54) COMPOSITIONS AND METHODS FOR TREATMENT OF TRAUMATIC BRAIN INJURY (TBI), FOR EXAMPLE, MILD TRAUMATIC BRAIN INJURY (mTBI)

(71) Applicant: THE SALLIE ASTOR BURDINE BREAST FOUNDATION, Baton Rouge, LA (US)

(72) Inventors: Brent M. Segal, Pembroke, MA (US); Catherine Baucom, Baton Rouge, LA (US); Xianpeng Jiang, Baton Rouge, LA (US)

(21) Appl. No.: 19/109,391

(22) PCT Filed: Sep. 8, 2023
(86) PCT No.: PCT/US2023/032292

§ 371 Date: Mar. 6, 2025

Related U.S. Application Data
(60) Provisional application No. 63/405,336, filed on Sep. 9, 2022.

(51) Int. Cl. A61K 35/33; A61K 9/00; A61K 47/02; A61K 47/18; A61K 47/20; A61K 47/22; A61K 47/26; A61K 47/42

(52) U.S. Cl. CPC A61K 35/33; A61K 9/0019; A61K 47/02; A61K 47/183; A61K 47/20; A61K 47/22; A61K 47/26; A61K 47/42

(57) ABSTRACT

Presented herein are compositions and methods involving mitochondrial organelle transplantation for use in the treatment of traumatic brain injury (TBI), e.g., mild traumatic brain injury (mTBI), in a subject. compositions and methods for treatment of traumatic brain injury (tbi), for example, mild traumatic brain injury (mtbi)

### CROSS-REFERENCE TO PENDING APPLICATION

This application claims priority to and the benefit of U.S. Provisional Patent Application No. 63/405,336, filed Sep. 9, 2022, the disclosure of which is incorporated by referenced herein in its entirety.

### FIELD

The subject matter described herein relates to compositions and methods for treatment of a patient. In certain embodiments, described herein are compositions and methods involving mitochondrial organelle transplantation for use in the treatment of traumatic brain injury (TBI), e.g., mild traumatic brain injury (mTBI), in a subject.

### BACKGROUND

Over a 10-year period from October 2001-2011, 1.6 million service members were deployed to Iraq and Afghanistan to fight the war on terrorism. Some estimates place the number of warfighters suffering from concussion to be between 5-35%. Injury from concussion, also referred to as mild Traumatic Brain Injury (mTBI), has been linked to cognitive impairment even one year later in 14% of the study participants. Another recent report by Cole et al. indicated that >50% of solders in the study reported “abnormally high” symptom levels upon “Return to Duty” and >50% reported at least one significant symptom after “Return to Duty.”

Therefore, there exists a need for new approaches to palliate post-concussion symptoms for warfighters who may be in austere environments.

### SUMMARY

Presented herein are compositions and methods involving mitochondrial organelle transplantation for use in the treatment of traumatic brain injury (TBI), e.g., mild traumatic brain injury (mTBI), in a subject.

Injuries resulting in traumatic brain injuries (TBIs) are a part of daily life. While some individuals may experience a TBI through a fall or a car accident, others work in professions that constantly expose individuals to TBIs. For example, those in the military and sports professions (e.g., football) are exposed constantly to scenarios that may result in a TBI. As described herein, TBIs (e.g., mTBIs) are often the result of a mechanical injury to the head of an individual. TBIs also include minor TBIs (mTBIs) (e.g., a concussion). After a TBI is experienced by an individual, they may experience many symptoms related to the TBI depending on its severity. Symptoms such as nausea, blurred vision, and constant headaches are common for those suffering from TBIs. Without wishing to be bound to any particular theory, Applicant submits that many symptoms related to TBIs are a result of mitochondrial damage and dysfunction. Mitochondrial damage and dysfunction can result in secondary injuries to the brain, thus causing many of symptoms associated with TBIs.

Applicant submits that many symptoms associated with TBIs can be treated via administration of mitochondria to a subject. As disclosed herein, Applicant has developed technologies, methods, and compositions for administering mitochondria to subjects. Based on Applicant's experiences with administering mitochondria to subjects suffering from neurodegenerative diseases, administration of healthy, allogeneic mitochondria will supplement mitochondria lost in a TBI and repair damaged mitochondria. Additionally, in certain embodiments, repairing and replacing the subject's mitochondrial will help in alleviating symptoms associated with TBIs.

Described herein are methods of treatment of TBI in a subject, including mild traumatic brain injury (mTBI). In certain embodiments, described herein are compositions and methods involving mitochondrial organelle transplantation for use in the treatment of TBI, e.g., mild traumatic brain injury (mTBI), in a subject.

In one aspect, the invention is directed to a method for allogeneic transplantation of mitochondria in a subject (e.g., a human subject) for treatment of traumatic brain injury (TBI) (e.g., mild traumatic brain injury (mTBI)), said method comprising administering to said subject a composition (e.g., a pharmaceutical composition) comprising mitochondria isolated from a donor other than the subject, wherein the subject has mTBI (e.g., has been diagnosed as having mTBI).

In certain embodiments, the composition further comprises a mitochondrial storing buffer having a potassium ion concentration safe for administration to humans (e.g., wherein said mitochondrial storing buffer comprises a pharmaceutically acceptable carrier).

In certain embodiments, the administering step comprises parenterally administering (e.g., by subcutaneous, intramuscular, or intravenous injection) at least one-unit dose of said composition to said subject. In certain embodiments, the administering step comprises both intramuscular injection and intravenous injection of said composition to said subject.

In certain embodiments, the method further comprises isolating said mitochondria from said donor. In certain embodiments, isolating said donor mitochondria comprises preparing cell lysate from tissue of the donor via tissue dissociation (e.g., using a bead-tube shake homogenizer). In certain embodiments, isolating said donor mitochondria comprises using a mitochondrial isolation buffer comprising a serine protease inhibitor (e.g., phenylmethylsulfonyl fluoride (PMFS)) (e.g., to prevent or reduce damage of donor mitochondria from digestive enzymes). In certain embodiments, the method comprises isolating said donor mitochondria without using an antibiotic.

In certain embodiments, the donor and the subject are not an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step)].

In certain embodiments, the composition administered to the subject does not comprise an antibiotic (e.g., and wherein the subject is not administered an antibiotic).

In certain embodiments, the composition comprises mitochondria isolated from human primary fibroblasts of the donor.

In certain embodiments, the method further comprises isolating the mitochondria from tissue of the donor (e.g., fibroblast mitochondria). In certain embodiments, the isolating step is conducting using a mitochondrial isolation buffer composition (e.g., the mitochondrial isolation buffer solution described herein). In certain embodiments, the mitochondrial isolation buffer composition comprises: a buffering agent; a chelating agent; a sugar; an agent that acts as a membrane stabilizer and/or oxygen radical scavenger and/or binder of Ca²+ and/or binder of free fatty acid; and a serine protease inhibitor. In certain embodiments, the composition does not comprise an antibiotic.

In certain embodiments, the method further comprises storing the isolated mitochondria at a temperature below −40° C. (e.g., below −60° C., below −70° C., or below −80° C., e.g., using liquid nitrogen).

In certain embodiments, the method comprises administering to the subject an iron-chelating agent (e.g., desferrioxamine or deferasirox).

In certain embodiments, the method comprises administering to the subject an antioxidant and/or a probiotic.

In another aspect, the invention is directed to a mitochondrial isolation buffer composition (e.g., an aqueous solution) for use in performing any of the methods described herein (e.g., for use in mitochondrial organelle transplantation), said composition comprising: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²+ and/or binder of free fatty acid (e.g., bovine serum albumin, BSA); and a serine protease inhibitor (e.g., phenylmethylsulfonyl fluoride (PMFS), also called phenylmethane sulfonyl fluoride) (e.g., wherein the composition further comprises isolated donor mitochondria, e.g., fibroblast mitochondria).

In certain embodiments, the composition does not comprise an antibiotic.

In another aspect, the invention is directed to a mitochondrial storing buffer composition (e.g., an aqueous solution) for use in performing any of the methods described herein (e.g., for use in mitochondrial organelle transplantation), said composition comprising: one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)┘ └e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²+ and/or binder of free fatty acid (e.g., bovine serum albumin, BSA) (e.g., wherein the composition further comprises isolated donor mitochondria, e.g., fibroblast mitochondria).

In certain embodiments, the composition does not comprise an antibiotic.

In another aspect, the invention is directed to a kit comprising a donor mitochondria composition (e.g., an aqueous composition) in a unit dosage effective to treat traumatic brain injury (TBI) (e.g., mild traumatic brain injury (mTBI)) in a subject, said donor mitochondria composition comprising: mitochondria isolated from tissue of a donor (e.g., fibroblast mitochondria); one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²+ and/or binder of free fatty acid (e.g., bovine serum albumin, BSA).

In certain embodiments, the donor and the subject are not an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step)].

In certain embodiments, the donor mitochondria composition does not comprise an antibiotic.

In certain embodiments, the kit further comprises instructions for optimizing the dose and/or frequency and/or route of administration of the composition.

It is contemplated that systems, devices, methods, and processes of the claimed invention encompass variations and adaptations developed using information from the embodiments described herein. Adaptation and/or modification of the systems, devices, methods, and processes described herein may be performed, as contemplated by this description.

In another aspect, the invention is directed to a method of treating traumatic brain injury (TBI), the method comprising: administering to a subject a composition comprising allogeneic mitochondria.

In certain embodiments, the subject has experienced a TBI (e.g., a fall, an explosion, a car accident, a shockwave, or other mechanical force).

In certain embodiments, the subject is suffering from a TBI symptom [e.g., a headache (e.g., that progressively gets worse and/or does not go away), repeated vomiting, nausea, convulsions (e.g., seizures), an inability to awaken from sleep, dilation of one or both pupils of the eyes, slurred speech, weakness and/or numbness in the extremities, loss of coordination, increased confusion, restlessness, and agitation].

In certain embodiments, the subject has experienced a mild traumatic brain injury (mTBI) (e.g., a concussion).

In certain embodiments, the subject is suffering from a mTBI symptom (e.g., lightheadedness, dizziness, blurred vision, tired eyes, ringing in the ears, a bad taste in the mouth, fatigue, lethargy, a change in sleep patterns, behavioral and/or mood changes, and trouble with memory, concentration, attention, and/or thinking).

In certain embodiments, the subject is administered the composition at the time of an injury that may result in a TBI (e.g., a mTBI).

In certain embodiments, the subject is administered the composition at after a period of time (e.g., an hour, a day, a week, a month, a year, 5 years) of a TBI (e.g., a mTBI) or a suspected TBI.

In certain embodiments, the TBI (e.g., mTBI) results in a loss of mitochondria in the subject.

In certain embodiments, the TBI (e.g., mTBI) results in mitochondrial injury dysfunction.

In certain embodiments, the method further comprises isolating mitochondria.

In certain embodiments, the method comprises isolating mitochondria from a donor (e.g., a healthy donor) other than the subject.

In certain embodiments, the method comprises isolating mitochondria from the subject.

In certain embodiments, the method further comprises creating a cell bank from a donor (e.g., a healthy donor) other than the subject.

In certain embodiments, the cell bank comprises fibroblasts (e.g., primary fibroblasts).

In certain embodiments, the cell bank comprises mesenchymal stromal cells (MSCs).

In certain embodiments, the method comprises obtaining a tissue biopsy (e.g., a skin tissue biopsy) from a donor.

In certain embodiments, the method comprises creating a tissue bank (e.g., from a biopsy, e.g., a skin tissue biopsy) (e.g., a frozen tissue bank) from a donor.

In certain embodiments, the composition further comprises a mitochondrial storing buffer having a potassium ion concentration safe for administration to humans.

In certain embodiments, the administering step comprises parenterally administering at least one-unit dose of said composition to the subject.

In certain embodiments, the administering step comprises both intramuscular injection and intravenous injection of said composition to said subject.

In certain embodiments, isolating the mitochondria comprises preparing cell lysate from tissue via tissue dissociation.

In certain embodiments, isolating the mitochondria comprises using a mitochondrial isolation buffer comprising a serine protease inhibitor.

In certain embodiments, a method further comprises isolating the mitochondria without using an antibiotic.

In certain embodiments, the subject and the donor from which mitochondria are obtained are not an HLA (human leukocyte antigen) match.

In certain embodiments, the composition administered to the subject does not comprise an antibiotic.

In certain embodiments, the isolating step is conducted using a mitochondrial isolation buffer composition.

In certain embodiments, the mitochondrial isolation buffer composition comprises: a buffering agent; a chelating agent; a sugar; an agent that acts as a membrane stabilizer and/or oxygen radical scavenger and/or binder of Ca²+ and/or binder of free fatty acid; and a serine protease inhibitor.

In certain embodiments, the mitochondrial isolation buffer composition does not comprise an antibiotic.

In certain embodiments, the method further comprises storing the isolated mitochondria at a temperature below −40° C.

In certain embodiments, the method further comprises administering to the subject an iron-chelating agent.

In certain embodiments, the method further comprises administering to the subject an antioxidant and/or a probiotic.

In certain embodiments, a method further comprises a mitochondrial isolation buffer composition for use, said composition comprising: a buffering agent; a chelating agent; a sugar; an agent that acts as a membrane stabilizer and/or oxygen radical scavenger and/or binder of Ca²+ and/or binder of free fatty acid; and a serine protease inhibitor.

In certain embodiments, the mitochondrial isolation buffer composition does not comprise an antibiotic.

In certain embodiments, a mitochondrial storing buffer composition comprises: one or more buffering agents; a source of magnesium ion; a chelating agent; a sugar; an antioxidant; a cytoprotective agent that binds to calcium ion; and an agent that acts as a membrane stabilizer and/or oxygen radical scavenger and/or binder of Ca²+ and/or binder of free fatty acid.

In certain embodiments, the mitochondrial storing buffer composition does not comprise an antibiotic.

In another aspect, the invention is directed to the use of allogeneic mitochondria for the treatment of traumatic brain injury (TBI) (e.g., mTBI) (e.g., in a method described herein) (e.g., using a composition described herein).

In another aspect, the invention is directed to the use of allogeneic mitochondria for the treatment of traumatic brain injury (TBI) (e.g., mTBI)(e.g., in a method described herein) (e.g., using a composition described herein), wherein the allogeneic mitochondria is administered to a subject suffering from traumatic brain injury (TBI) (e.g., mTBI)-associated mitochondrial damage.

In certain embodiments, the subject has experienced a TBI (e.g., a mTBI) (e.g., a fall, an explosion, a car accident, a shockwave, or other mechanical force).

In certain embodiments, the subject is suffering from a TBI symptom [e.g., a headache (e.g., that progressively gets worse and/or does not go away), repeated vomiting, nausea, convulsions (e.g., seizures), an inability to awaken from sleep, dilation of one or both pupils of the eyes, slurred speech, weakness and/or numbness in the extremities, loss of coordination, increased confusion, restlessness, and agitation].

In certain embodiments, the subject has experienced a mild traumatic brain injury (mTBI) (e.g., a concussion).

In certain embodiments, the subject is suffering from a mTBI symptom (e.g., lightheadedness, dizziness, blurred vision, tired eyes, ringing in the ears, a bad taste in the mouth, fatigue, lethargy, a change in sleep patterns, behavioral and/or mood changes, and trouble with memory, concentration, attention, and/or thinking).

In certain embodiments, the use comprises administering (e.g., intravenously, intramuscularly, subcutaneously) (e.g., both intravenously and intramuscularly) a composition (e.g., a composition described herein) comprising the allogeneic mitochondria the time of an injury that may result in a TBI (e.g., a mTBI).

In certain embodiments, the use comprises administering a composition (e.g., a composition described herein) comprising the allogeneic mitochondria after a period of time (e.g., an hour, a day, a week, a month, a year, 5 years) of a TBI (e.g., a mTBI) or a suspected TBI.

In certain embodiments, the subject is suffering from a loss of mitochondria.

In certain embodiments, the subject is suffering from mitochondrial injury dysfunction.

### DEFINITIONS

A or An: The articles “a” and “an” are used herein to refer to one or to more than one (i.e., to at least one) of the grammatical object of the article. By way of example, “an element”refers to one element or more than one element.
Administration: As used herein, the term “administration” typically refers to the administration of a composition to a subject or system, for example to achieve delivery of an agent that is, is included in, or is otherwise delivered by, the composition. Non-limiting examples of administration include oral administration; parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.
Agent: As used herein, the term “agent” refers to an entity (e.g., for example, a cell, a component of a cell such as mitochondria or other organelle, a small molecule, a peptide, a polypeptide, a nucleic acid, a lipid, a polysaccharide, a complex, a combination, a mixture, a system, or a phenomenon such as heat, electric current, electric field, magnetic force, magnetic field, etc.).
Allogeneic: As used herein, the term “allogeneic” refers to any material derived from one subject (a donor) and transplanted to another subject. Examples of allogeneic transplantation include allogeneic T cell transplantation, allogeneic stem cell transplantation, and, as discussed herein, allogeneic mitochondrial transplantation.
Amelioration: As used herein, the term “amelioration” refers to the prevention, reduction, palliation, or improvement of a state of a subject. Amelioration includes, but does not require, complete recovery or complete prevention of a disease, disorder or condition.
Antibiotic: As used herein, the term “antibiotic” refers to an antibacterial substance such as penicillin, gentamicin, streptomycin, cephalosporin, ciprofloxacin, or the like, that is used to treat or prevent infections by killing or inhibiting the growth of bacterial in or on the body, that is administered orally, topically, or by injection, and that is isolated from cultures of certain microorganisms (such as fungi) or is of semi-synthetic or synthetic origin.
Autologous: As used herein, the term “autologous” refers to any material derived from one subject (a donor) and transplanted back into that same subject. Herein “autologous” and “autogenic” are used interchangeably. Examples of autologous transplantation include autologous stem cell transplantation and autologous stem cell transplantation, and, as discussed herein, autologous mitochondrial transplantation.
Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or include biological tissue or fluid. In some embodiments, e.g., as set forth herein, a biological sample can be or include cells, tissue (e.g., skin tissue, muscle, or other tissue), or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or bronchioalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source, or can be a “processed sample.” A biological sample can also be referred to as a “sample.”
Improved, increased, or reduced: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.
Isolated: As used herein, “isolated” refers to a substance and/or entity (e.g., including one or more mitochondria) that has been (a) separated from at least some of the components with which it was associated when initially produced (whether in nature, in a subject such as a donor, and/or in an experimental setting), and/or (b) designed, produced, prepared, and/or manufactured by the hand of man. Isolated substances and/or entities may be separated from at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated substances and/or entities are at least about 80%, about 85%, about 90%, about 91%, about 92%, about 93%, about 94%, about 95%, about 96%, about 97%, about 98%, about 99%, or more than about 99% pure. As used herein, a substance and/or entity is “pure” if it is substantially free of other components. In some embodiments, as will be understood by those skilled in the art, a substance and/or entity may still be considered “isolated” or “pure” after having been combined with certain other components such as, for example, one or more carriers or excipients (e.g., buffer, solvent, water, etc.); in such embodiments, percent isolation or purity of the substance and/or entity is calculated without including such carriers or excipients. To give but one example, in some embodiments, mitochondria that occur in nature can be referred to as “isolated” when, (a) they are present in a composition that does not include some or all of the components with which they were associated in nature, e.g., in a donor from which they were derived; (b) they are substantially free of other organelles of a donor organism from which they were derived; (c) they are present in a cell or system that differs from the donor organism from which they were derived. Thus, for instance, mitochondria removed from a donor for transplantation into a second different subject can be referred to as “isolated.”
Neurodegenerative disease: As used herein, the term “neurodegenerative disease” (also referred to as “degenerative nerve disease”) is an umbrella term for conditions which primarily affect the neurons in the human brain. In certain instances, neurodegenerative disease is characterized by a progressive loss of neurons associated with deposition of proteins showing altered physicochemical properties in the brain and/or in peripheral organs. Neurodegenerative diseases include, for example, amyotrophic lateral sclerosis (ALS), Parkinson's disease (PD) and PD-related disorders, Alzheimer's disease (AD), Lewy body dementia (LBD), other forms of dementia, muscular dystrophy (MD), mitochondrial disorders, prion diseases, motor neuron diseases (MND), Huntington's disease (HD), multiple sclerosis (MS), spinocerebellar ataxia (SCA), spinal muscular atrophy (SMA), Friedreich's ataxia, Batten disease, fatal familial insomnia, and others.
Pharmaceutical composition: As used herein, the term “pharmaceutical composition” refers to a composition in which an active agent is provided together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenched (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.
Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.
Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.
Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.
Prognosis: As used herein, the term “prognosis” refers to determining the qualitative or quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.
Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.
Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample).
Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status associated with, development of the disease, disorder, or condition. In some embodiments, e.g., as set forth herein, a risk of developing a disease, disorder, and/or condition is a population-based risk (e.g., family members of individuals suffering from the disease, disorder, or condition).
Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”
Syngeneic: As used herein, the term “syngeneic” refers to any material derived from one subject (a donor) and transplanted to a subject wherein the subject and donor are genetically identical. Examples of syngeneic transplantation include syngeneic T cell transplantation, syngeneic bone marrow transplantation, and, as discussed herein, syngeneic mitochondrial transplantation.
Therapeutic agent, pharmaceutical agent, and active agent: As used herein, the terms “therapeutic agent”, “pharmaceutical agent”, and “active agent” are interchangeable, and each refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.
Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.
Treatment: As used herein, the term “treatment” (also “treat” or “treating”) refers to administration of a therapy that partially or completely alleviates, ameliorates, relieves, inhibits, delays onset of, halts progression of, slows progression of, reverses progression of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.
Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one-unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts
Variant: As used herein, the term “variant” refers to an entity that shows significant structural identity with a reference entity but differs structurally from the reference entity in the presence, absence, or level of one or more chemical moieties as compared with the reference entity. In some embodiments, e.g., as set forth herein, a variant also differs functionally from its reference entity. In general, whether a particular entity is properly considered to be a “variant” of a reference entity is based on its degree of structural identity with the reference entity. A variant can be a molecule comparable, but not identical to, a reference. For example, a variant peptide can differ from a reference peptide at one or more differences in the amino acid sequence. In some embodiments, e.g., as set forth herein, a variant peptide shows an overall sequence identity with a reference peptide that is at least 85%, 86%, 87%, 88%, 89%, 90%, 91%, 92%, 93%, 94%, 95%, 96%, 97%, or 99%.
Xenogeneic: As used herein, the term “xenogeneic” refers to any material derived from one subject (a donor) and transplanted to a subject wherein the subject and donor are different species. Examples of xenogeneic transplantation include xenogeneic skin transplantation, xenogeneic heart valve transplantation, and, as discussed herein, xenogeneic mitochondrial transplantation.

### DESCRIPTION OF THE INVENTION

Described herein are methods of treatment of traumatic brain injury (TBI), including mild traumatic brain injury (mTBI) by applying the methods and compositions described below.

International (PCT) Patent Application No. PCT/US2020/047359, filed Aug. 21, 2020, and published as International Publication No. WO 2021/141637 describes compositions and methods for treatment of amyotrophic lateral sclerosis (ALS), other neurodegenerative diseases (ND), and other mitochondrial disorders. In particular, described therein are experimental examples demonstrating mitochondrial organelle transplantation™ (MOT™) for the treatment of NDs such as ALS. This application incorporates by reference U.S. patent application Ser. No. 16/937,388, filed on Jul. 23, 2020, and U.S. Provisional Ser. No. 62/958,592 , filed Jan. 8, 2020. The contents of each of the above-referenced patent applications are incorporated by reference herein in their entirety. Moreover, all publications mentioned herein are incorporated by reference herein in their entirety.

Hubbard et al. studied the impact that altered mitochondrial bioenergetics had in a mild closed head injury (CHI) mouse model. The study established that mTBI results in mitochondrial dysfunction. Targeting this dysfunction with an infusion of healthy allogeneic mitochondria is key to reduction of mTBI symptoms stemming from mitochondrial damage. With several reports of intercellular mitochondrial transfer as a means of damaged tissue repair, mitochondrial replacement provides a viable route to rescue damaged cells following mTBI.

Using data and knowledge obtained from ongoing studies to treat neurodegenerative diseases, specifically ALS and Parkinson's disease, it can be deduced that MOT™ may reduce the severity of symptoms of mTBI and the length of time needed for recovery, facilitating quicker and more complete “Return to Duty” (e.g., for military professionals).

As of the filing of the instant specification, the MOT™ for treatment of ALS is moving towards an IND submission for a Phase I clinical trial. The MOT™ material is currently TRL 4 (Technology Readiness Level 4). Applicant has previously published on traumatic brain injury (TBI) in the mouse with controlled cortical injury (CCI), closed head TBI, and mild repetitive TBI (rmTBI) models. In addition, Applicant has published extensively on rodent models of stroke, spinal cord injury (SCI), ALS and other models of injury in rodents. In addition, Applicant and others have reported that TBI reduces mitochondrial respiration, enhances production of ROS, and triggers apoptotic cell death, suggesting a prominent role of mitochondria in TBI pathophysiology. The major cause of TBI-associated brain damage is secondary injury, which includes mitochondrial injury dysfunction. Mitochondrial dysfunction impairs hippocampal development and leads to loss of NSCs and adult neurogenesis. Genetic diseases that result in mitochondrial abnormalities often show progressive clinical signs of impaired cognitive function as well. The complex mitochondrial dysfunction after TBI requires treatment that specifically addresses the secondary injury(ies). Upon conclusion of an upcoming study, MOT™ for the mTBI recovery indication will be TRL 5.

### A. Mitochondria

In eukaryotic cells, mitochondria are known as the powerhouse, which generates adenosine triphosphate (ATP), by oxidative phosphorylation (OXPHOS). They also play an important role in synthesis of iron-sulfur clusters and heme, β-oxidation of fatty acids, homeostasis of calcium, iron and reactive oxygen species (ROS). Mitochondria are of particular importance in neurons. Neurons have high metabolic requirements—the brain consumes 20% of the body's resting ATP production despite being only 2% of its mass. Moreover, mitochondria are essential calcium buffering organelles in neurons that modulate local calcium dynamics, for example, regulate neurotransmitter release. Neurons are long-lived cells that persist throughout the lifespan of the individual and as such are more susceptible to the accumulating damage arising from mitochondrial dysfunction. Severe mitochondrial dysfunction comes in many forms, including defective OXPHOS, excessive ROS, impaired calcium buffering capacity, and defective mitochondrial dynamics In eukaryotic cells, mitochondria generate ATP by oxidative phosphorylation (OXPHOS) in the presence of oxygen. Mitochondria also play an important role in synthesis of iron-sulfur (Fe—S) clusters, β-oxidation of fatty acids, synthesis of heme prosthetic groups, the urea cycle, as well as homeostasis of calcium, iron and reactive oxygen species (ROS). Mitochondria are highly dynamic organelles which frequently fuse and divide. Mitochondrial fusion/fission allow segregation of damaged mitochondria, mitophagy to remove damaged mitochondria, and ultimately cell death if the damage is too severe. In addition, mitochondria can transfer between cells. Cells may be able to obtain functional mitochondria from other cells in order to satisfy their bioenergetics and biosynthetic needs. Without wishing to be bound to any particular theory, the possible mechanisms include tunneling nanotubes, extracellular vesicles and partial or complete cell fusion.

Mitochondrial dysfunction contributes to many diseases such as neurodegenerative disease, cardiac disease, and cancer. Mitochondrial dysfunction broadly includes states in which mitochondria of a cell, tissue, organism, or sample thereof, are characterized by (1) a decreased rate, amount, or efficiency of ATP production; (2) a decreased mitochondrial membrane potential; (3) a decreased number or concentration of mitochondria; and/or (4) an increased rate or amount of ROS production, relative to a reference. In some embodiments, a reference is a measurement or value representative of a healthy subject (e.g., a comparable subject with typical mitochondrial function and/or without a diagnosed medical condition known to impact mitochondrial function). In some embodiments, a reference is a measurement or value representative of a population of healthy subjects. In some embodiments, a reference is a measurement or value representative of the subject at an earlier time. Methods and techniques for measuring mitochondrial ATP production, mitochondrial membrane potential, number or concentration of mitochondria, and/or ROS production are known in the art.

Mitochondrial dysfunction has been documented in amyotrophic lateral sclerosis (ALS), Alzheimer's disease (AD), and Parkinson's disease (PD), for example. Mitochondria are essential for neural function because neurons highly depend on aerobic OXPHOS in mitochondria for their energetic needs. Defective mitochondrial respiration and ATP production in neurons result in neural dysfunction and degeneration. Mitochondria also produce ROS. If oxidative stress of ROS overwhelms the antioxidative defense most from superoxide dismutase (SOD), catalase (CAT) and glutathione peroxidase (GPX), ROS causes protein, lipid and DNA damage of neurons. In addition, overload of mitochondrial calcium and iron impairs ATP production and structures of mitochondria and neurons.

Mitochondria are highly present in cardiac cells due to the increased energy demands of such cells. Mitochondrial dysfunction is associated with the development of numerous cardiac diseases such as atherosclerosis, ischemia-reperfusion injury, hypertension, cardiac hypertrophy and heart failure.

Defects in mitochondrial function have also been linked to tumorigenesis. For example, it has been observed that cancer cells have an increase in glycolysis and lactate production in the presence of oxygen without an increase in OXPHOS, known as the “Warburg Effect”. Many cancers have mitochondrial defects and dysfunction. Glycolytic inhibitors have been found to suppress tumor growth in animal models and clinical trials.

### B. Traumatic Brain Injury

In certain embodiments, the methods and compositions described herein are used in treatment of a subject suffering from a traumatic brain injury (TBI) or symptoms related to a traumatic brain injury (TBI). Without wishing to be bound to any particular theory, a TBI can cause damage to and/or loss of mitochondria, which can result in secondary injuries to the brain. Secondary injuries due to mitochondrial damage (e.g., dysfunction) or loss can cause symptoms related to a TBI.

In certain embodiments, a TBI is as caused by a mechanical injury to the head from an external force (e.g., a fall, an explosion, a car accident, a shockwave, or other mechanical force). In certain embodiments, a mechanical injury to the head includes a bump, blow, and/or jolt to the head, and/or penetrating head injury.

In certain embodiments, a TBI can result in one or more symptoms including, but not limited to, a headache (e.g., that progressively gets worse and/or does not go away), repeated vomiting, nausea, convulsions (e.g., seizures), an inability to awaken from sleep, dilation of one or both pupils of the eyes, slurred speech, weakness and/or numbness in the extremities, loss of coordination, increased confusion, restlessness, and agitation.

In certain embodiments, a TBI is a mild traumatic brain injury (mTBI) (e.g., a concussion). In certain embodiments, an mTBI can result in one or more symptoms including, but not limited to, lightheadedness, dizziness, blurred vision, tired eyes, ringing in the ears, a bad taste in the mouth, fatigue, lethargy, a change in sleep patterns, behavioral and/or mood changes, and trouble with memory, concentration, attention, and/or thinking.

In certain embodiments, the severity of a TBI is determined at or near the time of the injury. In certain embodiments, the severity of a TBI is based on one or more factors including, but not limited to, the length of the loss of consciousness, the length of either memory loss or disorientation, and how responsive the individual was after the injury.

In certain embodiments, a treatment of TBI can be administered at or after a period of time after an initial injury (e.g., immediately after an injury, an hour after the injury, a day after the injury, a week after the injury, a month after the injury, a year after the injury, 5 years after the injury).

### C. Selection of Tissue Donors

Where allogeneic mitochondria are used (e.g., from a donor other than the subject receiving the mitochondria), prior to donation of a tissue (e.g., skin) for use in creating a cell bank (e.g., a fibroblast cell bank), a donor is screened. In certain embodiments, screening a donor ensures that they are suitable for donation of tissue (e.g., skin tissue) from which cells are obtained. Mitochondria are isolated from cells obtained from a donor.

In certain embodiments, a donor is selected as required by FDA 21 CFR Part 1271, which is incorporated by reference in its entirety. In certain embodiments, a donor is screened for one or more diseases (e.g., communicable diseases, e.g., a virus). In certain embodiments, a donor is free from risk factors for, and clinical evidence of, infection due to a relevant communicable disease agent and/or disease. In certain embodiments, a donor is free from communicable disease risks associated with xenotransplantation. In certain embodiments, a donor is tested for communicable disease agents and is determined to be negative or nonreactive. In certain embodiments, a donor is free from diseases including, but not limited to, human immunodeficiency virus (HIV) (e.g., HIV1, HIV2, HBV, HCV), hepatitis B virus, hepatitis C virus, human transmissible spongiform encephalopathy (e.g., Creutzfeldt-Jakob disease), Treponema pallidum, and communicable diseases of the genitourinary tract (e.g., Chlamydia trachomatis, Neisseria gonorrhea).

In certain embodiments, a donor does not have a neurodegenerative disease and/or other condition associated with mitochondrial dysfunction. In certain embodiments, a donor does not have a disease including, but not limited to, amyotrophic lateral sclerosis (ALS), Parkinson's disease (PD), a PD related disorder, Alzheimer's disease (AD), Lewy body dementia (LBD), dementia, muscular dystrophy (MD), a mitochondrial disorder, prion disease, motor neurone disease (MND), Huntington's disease (HD), multiple sclerosis (MS), spinocerebellar ataxia (SCA), spinal muscular atrophy (SMA), Friedreich's ataxia, Batten disease, and fatal familial insomnia.

In certain embodiments, a donor does not have a history of cancer, diabetes or genetic mitochondrial diseases. In certain embodiments, a donor is not pregnant or a minor or adult individual who lacks capacity to consent.

In certain embodiments, a donor is from 18 yrs old to 40 yrs old. In certain embodiments, a donor is older than 18 years old. In certain embodiments, a donor is younger than 40 years old (e.g., younger than 30 years old, younger than 25 years old, younger than 20 years old).

Mitochondria derived from primary fibroblasts exhibit low or no immunogenicity and, accordingly, need no HLA tying match in mitochondrial transplantation. Humans have three main MHC class I loci, known as HLA-A, HLA-B, and HLAC, each individual carrying two alleles at each locus. Humans have six main MHC class II loci, known as HLA-DPA1, HLA-DPB1, HLA-DQA1, HLA-DQB1, HLA-DRA, and HLA-DRB1, each individual carrying two alleles at each locus. In general, a donor and a subject would be matched based on alleles present in the donor and the subject at one or more HLA loci, such as HLA-A, HLA-B, HLA-C, HLA-DRB1, HLA-DQB1, and/or HLA-DPB1. Various standards for HLA matching are known in the art. Matching of all 8 alleles at HLA-A, HLA-B, HLA-C, and HLA-DRB1 loci can be referred to as an 8/8 match. Matching of all 10 alleles at HLA-A, HLAB, HLA-C, HLA-DRB1, and HLA-DQB1 loci can be referred to as a 10/10 match. For certain transplantations, varying degrees of allele mismatch can be accepted. Thus, for example, a donor and a subject can be matched, e.g., at 10, 9, 8, 7, 6, 5, 4, 3, 2, 1, or 0 alleles of 10 at HLA-A, HLA-B, HLA-C, HLA-DRB1, and HLA-DQB1, or at 8, 7, 6, 5, 4, 3, 2, 1, or 0 alleles of 8 HLAA, HLA-B, HLA-C, and HLA-DRB1.

As is disclosed herein, in various embodiments, the donor and the subject for mitochondrial transplantation do not need to be an HLA (human leukocyte antigen) match [e.g., not an identical match (e.g., based on match of 8 or 10 tested HLA markers) and/or not a haploidentical match (e.g., based on match of 8 or 10 tested HLA markers), and/or of indeterminate match status (e.g., no HLA markers tested prior to the administering step). This is a significant advantage over stem cell therapy and organ transplantation which require HLA matching of donor and recipient.

### D. Obtaining Cells From Donor Tissue

In some embodiments, a cell bank is created from cells of a donor (e.g., a donor different from the subject receiving a mitochondrial composition). In some embodiments, a cell bank is a frozen cell bank. In some embodiments, cells are stored in a cell bank by freezing. In certain embodiments, cells are frozen at a temperature below −40° C. (e.g., below −60° C., below −70° C., below −80° C., e.g., using liquid nitrogen). In certain embodiments, medium used to freeze cells to create a cell bank does not contain antibiotics.

In some embodiments, skin tissue is used as source material to create a cell bank. In certain embodiments, fibroblast cells from skin tissue are obtained to create a cell bank. In certain embodiments, fibroblast cells from skin tissue are expanded prior to freezing. In certain embodiments, fibroblast cells from skin tissue are expanded in a culture media that is not supplemented with antibiotics (e.g., gentamicin, penicillin, streptomycin). Antibiotics in culture media can be toxic to mitochondria of mammalian cells. In certain embodiments, fibroblast cells from a first, a second, or a third passage are frozen to create a cell bank.

In certain embodiments, mesenchymal stem cells (MSCs) (e.g., primary MSCs) are obtained from a donor (e.g., for creating a cell bank).

A person of skill in the art would be able to select appropriate freezing protocols corresponding to tissue and cell types described herein based on information provided in the present disclosure.

### E. Mitochondrial Isolation and Storing Buffers

In certain embodiments, the methods and compositions described herein utilize isolation and storing buffers for mitochondria. In certain embodiments, mitochondrial isolation and storing buffers used are described in International (PCT) Patent Application No. PCT/US2020/047359, filed Aug. 21, 2020, which is incorporated by reference in its entirety.

In certain embodiments, a mitochondrial isolation buffer (e.g., for isolation of mitochondria) comprises a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²+ and/or binder of free fatty acid (e.g., bovine serum albumin, BSA); and a serine protease inhibitor (e.g., phenylmethylsulfonyl fluoride (PMFS), also called phenylmethane sulfonyl fluoride). In certain embodiments, the composition further comprises isolated donor mitochondria, e.g., fibroblast mitochondria.

In certain embodiments, a mitochondrial isolation buffer is comprised of 300 mM sucrose, 10 mM K-HEPES, 1 mM K-EGTA, 0.1% BSA and 0.25 mM PMSF (Sigma Aldrich, St Louis, MO, USA). In certain embodiments, the osmolarity of the buffer is about 325 mOsm. In certain embodiments, the concentration of potassium ion is 11 mM. Bovine serum albumin (BSA) is a membrane stabilizer, oxygen radical scavenger, and binds Ca²+ and free fatty acids. Phenylmethylsulfonyl fluoride (PMSF), also called phenylmethane sulfonyl fluoride, is a serine protease inhibitor used in the preparation of cell lysates. Lysosomes are organelles that contain digestive enzymes which digest excess or worn out organelles. During the procedure of cell homogenization (e.g., to obtain mitochondria), some lysosomes may be damaged and release the digestive enzymes to the cell lysate. In certain embodiments, in order to prevent the damage of mitochondria from the digestive enzymes, PMSF can be included in the isolation buffer. In certain embodiments, the isolation buffer does not contain antibiotics.

In certain embodiments, a mitochondrial storing buffer is administered to a subject and/or used to maintain isolated mitochondria stably in solution. In certain embodiments, a storage buffer comprises one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)] [e.g., monopotassium phosphate (KH₂PO₄)]; a source of magnesium ion [e.g., magnesium chloride (MgCl₂)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²+ and/or binder of free fatty acid (e.g., bovine serum albumin, BSA). In certain embodiments, the composition further comprises isolated donor mitochondria (e.g., fibroblast mitochondria). In certain embodiments, the storing buffer does not contain antibiotics.

In certain embodiments, a mitochondrial storing buffer is comprised of 240 mM sucrose, 2 mM KH₂PO₄, 3 mM MgCl₂, 10 mM K-HEPES, 20 mM Taurine, 1 mM K-EGTA, 0.1% BSA and 15 mM K-lactobionate (Sigma Aldrich, St Louis, MO, USA). Taurine acts as an antioxidant that scavenges free radical species generated by mitochondria, and is also involved to membrane stabilization, osmoregulation and ion channel regulation. Lactobionate has cytoprotective property and prevents mitochondrial swelling. Lactobionate also binds to calcium ion with high affinity and acts as a calcium chelator. In certain embodiments, the osmolarity of a mitochondrial storing buffer is about 325 mOsm. In certain embodiments, a mitochondrial storing buffer contains about 28 mM potassium ion. In certain embodiments, buffers are sterilized by filtering (e.g., through a 0.22 μm filter), aliquoted to small vials and stored at −80° C.

In certain embodiments (e.g., clinical uses discussed herein), mitochondria in the storing buffer is administered (e.g., intramuscularly, intravenously, subcutaneously) (e.g., both intramuscularly and intravenously) to a human subject. High potassium ion concentrations are dangerous for injection to humans (e.g., 91 mM potassium ion concentration). In certain embodiments, a reduced concentration of all K⁺ salts is used in a mitochondrial storing buffer (e.g., 2 mM KH₂PO₄, 10 mM K-HEPES, and 15 mM K-lactobionate). In certain embodiments, the final concentration of K⁺ in a storing buffer solution used is similar to clinical intravenous solution with potassium chloride (e.g., about 28 mEq) (e.g., from 20 mEq to 40 mEq). To keep the osmolarity of the storing buffer at a desired level, the concentration of sucrose can be increased in the storing buffer. In certain embodiments, the storing buffer is not administered along with antibiotics.

### F. Description of Upcoming Study, Scientific Rationale, and Initial Data

The scope of this effort with a VA partner is to test efficacy of administration by infusion of MOT™ to reduce time to recovery and alleviate severity of symptoms in mTBI animal models. Using key findings from neurodegeneration in ALS, Parkinson's, and knowledge from brain injury models from the VA, Applicant hypothesizes that mitochondrial dysfunction is responsible for many of the symptoms associated with mTBI. Accordingly, it is thought that infusion of healthy allogeneic mitochondria via transplantation will supplement mitochondria lost in injury and repair mitochondria that were damaged; and that the infusion will result in objective amelioration of mTBI in the models. During the course of these studies, information will be gained confirming safety in the specific context of mTBI and manufacturing improvements focused on shipping to and use in austere environments will be investigated. Successful completion of these studies will enable submission of an investigational new drug (IND) application to FDA, thus paving the way for the therapeutic use of MOT™ to reduce the time needed for a Service Member's “Return to Duty” and reduce the severity of Service Member symptoms after they “Return to Duty”.

In recent years, mitochondrial transplantation (MT) has emerged as a promising therapeutic intervention that benefits neuronal survival and regeneration for neurodegenerative diseases, stroke, and central nerve system (CNS) injury. McCully's group have extensively studied the role of injected isolated mitochondria for cardio protection during ischemia-reperfusion. The exogenous mitochondria could enter cardiomyocytes at 2 hours after injection and maintained viability and function producing adequate ATP levels. They also demonstrated that the exogenous mitochondria provided cardio protection both extracellularly and intracellularly. Shi et al, intravenously injected xenogeneic mitochondria from human hepatoma into the experimental Parkinson's disease (PD) model rat. The MT prevented experimental PD progress through increasing activity of electron transport chain, decreasing ROS level, and preventing cell apoptosis and necrosis. In 2016, Huang group intracerebrally or intra-arterially injected xenogeneic mitochondria of hamster cells to brain ischemic rat model. Their results confirmed that hamster mitochondria transfer protected neural function and promotes functional recovery in brain ischemic rats. They found that the mitochondrial internalization to neurons could not completely account for the high rescue of neuronal injury.

Extracellular, exogenous mitochondria may be a source of ATP and a ROS scavenger to protect cells from damage by free radicals. Applicant has reported that human mitochondria from primary fibroblasts transplant into rat motor neuronal NSC-34 mitochondrial DNA (mtDNA)-depleted (ρ⁰) cells. MT replenishes mtDNA and restores mitochondrially encoded cytochrome c oxidase 1 (MT-CO1) and mitochondrially encoded NADH dehydrogenase 1 (MT-ND1) and rescues aerobic respiration of NSC-34 ρ⁰ cells. Moreover, Applicant has found that isolated human mitochondria do not contain detectible human leukocyte antigen I (HLA-I) which suggests allogeneic mitochondria exhibiting low or no immunogenicity and needing no HLA-tying match in MT.

Mesenchymal stem/stromal cell (MSC) therapy has also received a great deal of attention.

Bidirectional mitochondrial transfer is observed between both donor and recipient cells and has an impact on both donor and recipient cells. Mitochondria transfer improves cell viability, promotes anti-inflammation, and facilitates differentiation of stem cells. By adding isolated normal mitochondria to induced pluripotent stem cells (iPSCs), isolated normal mitochondria enter stem cells within minutes and facilitate the differentiation into neurons, by increased expression of neuronal and glutamatergic markers β3-tubulin and synapsin1, and by an activation of the glutamate-glutamine cycle. MOT™ could replenish mitochondria and mtDNA and restore mitochondrial function of defective neurons in mTBI. Gollihue et al. reported that transplantation of exogenous mitochondria maintained acute bioenergetics of the injured spinal cord in a concentration-dependent manner. Elliott et al. published a case of ALS patient treated by MOT™ under Compassionate Care Act. The patient participated in two previous clinical trials, but her disease continuously progressed, and her condition declined. After 4 transplant procedures at 6 weeks intervals, the patient had no adverse reactions or complications but had significant improvement in leg muscle strength via testing conducted and clinical function measured. Also, the patient regained all sensory sensations in both legs.

The MOT™ protocol uses fibroblasts from a healthy donor. The cells were stored in liquid nitrogen in the Elliott MRC laboratory. The fibroblasts were recovered from liquid nitrogen and expanded in a GLP cell culture facility. The mitochondria were then isolated from the fibroblasts in a GLP cell culture facility. The isolation protocol is simple and fast, and maintains good mitochondrial yield, viability, and quality. The mitochondria can be injected intramuscularly and intravenously.

Applicant has conducted a pilot study of repeated intramuscular and intravenous administration of MOT™ in a series of 5 patients with ALS during the prior 2.5 years, and have not observed any adverse events, in a total of 47 administrations. These patients were followed for a period of 45-90± days between treatment cycles, and although this small sample is not powered to detect efficacy, four patients reported symptom improvement noted over several weeks following administration. This real-world data provided the basis for the Human Experience component of IND application to FDA. Applicant has communicated with FDA leadership (Dr. Peter Marks), who has confirmed that MOT™ will be appropriately regulated via the IND pathway within the Center for Biologics Evaluation and Research (CBER).

In support of the above clinical preliminary data, Applicant has successfully manufactured clinical-grade batches of MOT™, which were used to provide doses for the case series. The MOT™ preparations showed consistency with respect to the wet weight of mitochondria. For this project, the team will provide GLP-grade batches of MOT™. The MOT™ material is preparing to achieve GMP production development for use in treating ALS. The methods for MOT™ are available to the team to enable progress from TRL 4 to TRL 5. The GMP processing of MOT™ material will be overseen by KRS Global Biotechnology, an industry-leading customized FDA registered and inspected 503b Human Outsourcing Facility, that provides sterile and non-sterile compounding services to patients, surgery centers, ophthalmology clinics, hospitals, and universities. In collaborating with KRS, Applicant will assess on shipping to and use of MOT™ in austere environments. Following successful conclusion of this study, it will be possible to move into clinical trials.

### G. Constructive Example

Healthy human mitochondria will improve motor function and recovery post-concussion (mTBI) of the animal mTBI models. First, human primary fibroblasts will be chosen from healthy donors to be used for MOT™. A primary fibroblast bank will be developed. Skin tissues will be donated by healthy volunteers, 18 to 40 years of age. Donors with infectious diseases (HIV1, HIV2, HBV, HCV and treponema pallidum), a history of cancer, diabetes or genetic mitochondrial diseases, pregnant women, and minor or adult individuals who lack capacity to consent for themselves are excluded from donation. Tissue collection protocol and consent form have been approved by Institutional Review Board (the WCG IRB [formerly Western] for the Elliott MRC at the SABBF). Isolation, culture, and cryopreservation of human primary fibroblasts was reported previously in Jiang et al. (Jiang X P, Baucom C C, Elliott R L. Mitochondria dynamically transplant into cells in vitro and in mice and rescue aerobic respiration of mitochondrial DNA-depleted motor neuron NSC-34. J. Biomedical Sci. Engineering. 2020; 13:203-221. doi: 10.4236/jbise.2020.139019). Second, primary fibroblasts will be recovered from the human fibroblast bank and expanded. Mitochondria will be isolated from the fibroblasts by differential centrifugation (e.g., according to Jiang et al.). Third, assays will be further developed to determine mitochondrial number, integrity, viability, and potency. Data from the assays will be used to refine the protocol of mitochondrial isolation and storage and for quality control parameters for MOT™in animal mTBI models.

Data from the assays will be used to refine the mitochondrial dosing for MOT™ in the animal mTBI models. Fourth, safety of mitochondria and buffer will be tested in different administration routes such as injection subcutaneously or intravenously. Fifth, mitochondrial distribution in tissues including spinal cord and brain will be examined by injection of fluorescence-labelled mitochondria. Sixth, efficacy including motor function and neurological behavior of mice and rats will be studied using previously reported biomarkers and assays (e.g., discussed in refs. 27 and 28). These biomarkers may allow prediction about the potential efficacy of the MOT™ treatment for mTBI treatment. Seventh, at the endpoint, pathological and ultrastructural studies will be completed for mouse and rat tissues including spinal cord, brain, and muscles to demonstrate the mechanism of MOT™. These studies will be repeated in a pig model after analysis of mouse and rat studies to confirm findings in a larger animal model. Finally, the data will be statistically analyzed, reported, and published in an appropriate peer-reviewed journal.

The proposed work will demonstrate the impact of MOT™ on reducing 1) recovery time in three animal mTBI models (mouse, rat, and pig), and 2) the severity of mTBI symptoms after injury in animal models. Success will validate MOT™ as a viable treatment for mTBI in reducing “Return to Duty” time and, once FDA approved, will be a strong addition to the DoD and Force Readiness.

### PUBLICATIONS

1. Rigg J L, Mooney S R. Concussions and the military: issues specific to service members. PM R. 2011 October; 3(10 Suppl 2): S380-6. doi: 10.1016/j.pmrj.2011.08.005. PMID: 22035680.
2. Christman Schneider, Andrea Lauren et al “Cognitive Outcome 1 Year After Mild Traumatic Brain Injury: Results From the TRACK-TBI Study.” Neurology (2022): 10.1212/WNL.0000000000200041. Web. 14 Mar. 2022.
3. Wesley R. Cole, Amy S. Cecchini, Rosemay A. Remigio-Baker, Emma Gregory, Jason M. Bailie, Mark L. Ettenhofer & Karen L. McCulloch (2020) “Return to duty” as an outcome metric in military concussion research: Problems, pitfalls, and potential solutions, The Clinical Neuropsychologist, 34:6, 1156-1174, DOI: 10.1080/13854046.2020.1715484
4. Hubbard W B, Joseph B, Spry M, Vekaria H J, Saatman K E, Sullivan P G. Acute Mitochondrial Impairment Underlies Prolonged Cellular Dysfunction after Repeated Mild Traumatic Brain Injuries. J Neurotrauma. 2019 Apr. 15; 36(8):1252-1263. doi: 10.1089/neu.2018.5990. Epub 2018 Dec. 20. PMID: 30417732; PMCID: PMC7207062.
5. Liu D, Gao Y, Liu J, Huang Y, Yin J, Feng Y, Shi L, Meloni BP, Zhang C, Zheng M, Gao J. Intercellular mitochondrial transfer as a means of tissue revitalization. Signal Transduct Target Ther. 2021 Feb. 16; 6(1):65. doi: 10.1038/s41392-020-00440-z. PMID: 33589598; PMCID: PMC7884415.
6. Gollihue J L, Patel S P, Eldahan K C, Cox D H, Donahue R R, Taylor B K, et al. Effects of Mitochondrial Transplantation on Bioenergetics, Cellular Incorporation, and Functional Recovery after Spinal Cord Injury. J Neurotrauma. 2018; 35(15): 1800-1818. doi: 10.1089/neu.2017.5605
7. Fang S Y, Roan J N, Lee J S, Chiu M H, Lin M W, Liu C C, Lam C F. Transplantation of viable mitochondria attenuates neurologic injury after spinal cord ischemia. J Thorac Cardiovasc Surg. 2021 May; 161(5):e337-e347. doi: 10.1016/j.jtcvs.2019.10.151.
8. Hook G R, Yu J, Sipes N, Pierschbacher M, Hook V, Kindy M. The Cysteine Protease Cathepsin B is an Important Drug Target and Cysteine Protease Inhibitors are Potential Therapeutics for Traumatic Brain Injury. J. Neurotrauma, 2014, 31(5):515-29.
9. Novgorodov S, Riley C L, Yu J, Borg K T, Hannun Y A, Proia R L, Kindy M, Gudz T. Essential Roles of Neutral Ceramidase and Sphingosine in Mitochondrial Dysfunction Due to Traumatic Brain Injury. J. Biol. Chem. 2014, 289(19):13142-54.
10. Hook G, Jacobson J S, Grabstein K, Kindy M, Hook V. Cathepsin B is a new drug target for traumatic brain injury therapeutic: Evidence for E64d as a lead drug candidate. Frontiers in Neurology, 2015, 6:178.
11. Novgorodov S A, Riley C L, Keffler J A, Yu J, Kindy M S, Macklin W B, Lombard D B, Gudz T I. SIRT3 Deacetylates Ceramide Synthases, Promoting Mitochondrial Dysfunction and Brain Injury. Journal of BC, J. Biol. Chem., 2016; 291:1957-1973.
12. Yu J, Zhu H, Taheri S, Mondy W, Perry S, Kindy M S. Impact of nutrition on inflammation, tauopathy and behavioral outcomes from chronic traumatic encephalopathy. J. Neuroinflammation, 2018:15(1), 277.
13. Yu J, Zhu H, Taheri S, Perry S, Kindy M S. Reduced neuroinflammation and improved functional recovery after traumatic brain injury by diet supplementation in mice. Nutrients., 2019; 11: E299.
14. Hoffman J, Badanich K, Yu J, Kirstein C, Kindy M S. Combined Effects of rmTBI and Alcohol Drinking on the Neuroinflammatory Cytokine Response and Cognitive Behavioral Outcomes. Brain Sciences. 2020; 10:876.
15. Zhao J, Qu D, Xi Z, Huan Y, Zhang K, Yu C, Yang D, Kang J, Lin W, Wu S, Wang Y. Mitochondria transplantation protects traumatic brain injury via promoting neuronal survival and astrocytic BDNF. Transl Res. 2021; 235:102-114.
16. Murphy E, et al. Mitochondrial function, biology, and role in disease. Circulation Research 2016; 118:1960-1991. doi.org/10.1161/RES.0000000000000104.
17. Smith E F, Shaw P J, De Vos K J. The role of mitochondria in amyotrophic lateral sclerosis. Neurosci Lett. 2019; 710:132933. doi: 10.1016/j. neulet.2017.06.052.
18. Federico A, et al. Mitochondria, oxidative stress and neurodegeneration. J Neurol Sci. 2012; 322:254-62. doi: 10.1016/j.jns.2012.05.030.
19. Elliott R L, Jiang X P. Neurodegeneration and mitochondria organelle transplantation: “A technology that proof of principle suggest is ready for prime time”. Neuroscience and Medicine 2020; 11:108-118. doi: 10.4236/nm.2020.114013.
20. Norat P, et al. Mitochondrial dysfunction in neurological disorders: Exploring mitochondrial transplantation. NPJ Regen Med 2020; 5:22. doi: 10.1038/s41536-020-00107-x.
21. Masuzawa A, et al Transplantation of autologously derived mitochondrial protects the heart form ischemia-reperfusion injury. Am. J. Physiology-Heart and Circulatory Physiology, 2012; 304: H966-H982. https://doi.org/10.1152/ajpheart.00883.2012.
22. Shi X, Zhao M, FuC, FuA. Intravenous administration of mitochondria for treating experimental Parkinson's. Mitochondrion 2017; 34:91-100.
23. Huang P J, et al. Transferring xenogenic mitochondria provides neural protection against ischemic stress in ischemic rat brains. Cell Transplantation 2016; 25:913-927. https://doi.org/10.3727/096368915X689785.
24. Jiang X P, Baucom C C, Elliott R L. Mitochondria dynamically transplant into cells in vitro and in mice and rescue aerobic respiration of mitochondrial DNA-depleted motor neuron NSC-34. J. Biomedical Sci. Engineering. 2020; 13:203-221. doi: 10.4236/jbise.2020.139019.
25. Deqiang Han, et al. Mesenchymal stem/stromal cell-mediated mitochondrial transfer and the therapeutic potential in treatment of neurological diseases. Stem Cells Int. Vol. 2020, Article ID 8838046. doi.org/10.1155/2020/8838046.
26. Robicsek O, et al. Isolated mitochondria transfer improves neuronal differentiation of Schizophrenia-derived induced pluripotent stem cells and rescues deficits in a rat model of the disorder. Schizophrenia Bulletin. 2018; 44:432-442. https://doi.org/10.1093/schbul/sbx077.
27. Ranganathan S, Williams E, Ganchev P, Gopalakrishnan V, Lacomis D, Urbinelli L, Newhall K, Cudkowicz M E, Brown R H Jr, Bowser R. Proteomic profiling of cerebrospinal fluid identifies biomarkers for amyotrophic lateral sclerosis. J Neurochem. 2005 December; 95(5):1461-71. doi: 10.1111/j.1471-4159.2005.03478. x. PMID: 16313519; PMCID: PMC1540444.
28. Barschke P, Oeckl P, Steinacker P, Ludolph A, Otto M. Proteomic studies in the discovery of cerebrospinal fluid biomarkers for amyotrophic lateral sclerosis. Expert Rev Proteomics. 2017 September; 14(9):769-777. doi: 10.1080/14789450.2017.1365602. Epub 2017 Aug. 14. PMID: 28799854.
29. Abbott receives FDA 510(k) clearance for the first rapid handheld blood test for concussions. Abbott MediaRoom. (n. d.). Retrieved Apr. 9, 2022, from https://abbott.mediaroom.com/2021-01-11-Abbott-Receives-FDA-510-k-Clearance-for-the-First-Rapid-Handheld-Blood-Test-for-Concussions
30. Gan Zoe S., Stein Sherman C., Swanson Randel, Guan Shaobo, Garcia Lizette, Mehta Devanshi, Smith Douglas H., Blood Biomarkers for Traumatic Brain Injury: A Quantitative Assessment of Diagnostic and Prognostic Accuracy. Frontiers in Neurology VOLUME=10; 2019; DOI=10.3389/fneur.2019.00446. ISSN=1664-229 5https://www.frontiersin.org/article/10.3389/fneur.2019.00446
31. Wang K K, Yang Z, Zhu T, Shi Y, Rubenstein R, Tyndall J A, Manley G T. An update on diagnostic and prognostic biomarkers for traumatic brain injury. Expert Rev Mol Diagn. 2018 February; 18(2):165-180. doi: 10.1080/14737159.2018.1428089. Epub 2018 Jan. 23. PMID: 29338452; PMCID: PMC6359936.
32. Ramon Diaz-Arrastia, Kevin K. W. Wang, Linda Papa, Marco D. Sorani, John K. Yue, Ava M. Puccio, Paul J. McMahon, Tomoo Inoue, Esther L. Yuh, Hester F. Lingsma, Andrew I. R. Maas, Alex B. Valadka, David O. Okonkwo, Geoffrey T. Manley and the TRACK-TBI Investigators, including Scott S. Casey, Maxwell Cheong, Shelly R. Cooper, Kristen Dams-O'Connor, Wayne A. Gordon, Allison J. Hricik, David K. Menon, Pratik Mukherjee, David M. Schnyer, Tuhin K. Sinha, and Mary J. Vassar. Acute Biomarkers of Traumatic Brain Injury: Relationship between Plasma Levels of Ubiquitin C-Terminal Hydrolase-L1 and Glial Fibrillary Acidic Protein; Journal of Neurotrauma 2014 31:1, 19-25.
33. MILD TRAUMATIC BRAIN INJURY TREATMENT MARKET-GROWTH, TRENDS, COVID-19 IMPACT, AND FORECASTS (2022-2027): https://mordorintelligence.com/industry-reports/mild-traumatic-brain-injury-treatment-markethttps://mordorintelligence.com/industry-reports/mild-traumatic-brain-injury-treatment-market

It is contemplated that systems, compositions, methods, and processes of the claimed invention encompass variations and adaptations developed using information from the embodiments described herein. Adaptation and/or modification of the systems, compositions, methods, and processes described herein may be performed, as contemplated by this description.

Throughout the description, where articles, compositions, and systems are described as having, including, or comprising specific components, or where processes and methods are described as having, including, or comprising specific steps, it is contemplated that, additionally, there are articles, compositions, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein.

Documents are incorporated herein by reference as noted. Where there is any discrepancy in the meaning of a particular term, the meaning provided in this document is controlling.

Headers are provided for the convenience of the reader-the presence and/or placement of a header is not intended to limit the scope of the subject matter described herein.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for allogeneic transplantation of mitochondria in a subject for treatment of traumatic brain injury (TBI), said method comprising administering to said subject a composition comprising mitochondria isolated from a donor other than the subject, wherein the subject has mTBI.

2. The method of claim 1, wherein the composition further comprises a mitochondrial storing buffer having a potassium ion concentration safe for administration to humans.

3. The method of claim 1, wherein the administering step comprises parenterally administering at least one-unit dose of said composition to said subject.

4. The method of claim 3, wherein the administering step comprises both intramuscular injection and intravenous injection of said composition to said subject.

5. The method of claim 1, further comprising isolating said mitochondria from said donor.

6. The method of claim 5, wherein isolating said donor mitochondria comprises preparing cell lysate from tissue of the donor via tissue dissociation.

7. The method of claim 5, wherein isolating said donor mitochondria comprises using a mitochondrial isolation buffer comprising a serine protease inhibitor.

8. The method of claim 5, comprising isolating said donor mitochondria without using an antibiotic.

9. The method of claim 1, wherein the donor and the subject are not an HLA (human leukocyte antigen) match.

10. The method of claim 1, wherein the composition administered to the subject does not comprise an antibiotic.

11. The method of claim 1, wherein the composition comprises mitochondria isolated from human primary fibroblasts of the donor.

12. The method of claim 1, further comprising isolating the mitochondria from tissue of the donor.

13. The method of claim 12, wherein the isolating step is conducting using a mitochondrial isolation buffer composition.

14. The method of claim 13, wherein the mitochondrial isolation buffer composition comprises:
a buffering agent;
a chelating agent;
a sugar;
an agent that acts as a membrane stabilizer and/or oxygen radical scavenger and/or binder of Ca²+ and/or binder of free fatty acid; and
a serine protease inhibitor.

15. The mitochondrial isolation buffer composition of claim 14, wherein the composition does not comprise an antibiotic.

16. The method of claim 12, further comprising storing the isolated mitochondria at a temperature below −40° C.

17. The method of claim 1, comprising administering to the subject an iron-chelating agent.

18. The method of claim 1, comprising administering to the subject an antioxidant and/or a probiotic.

19. A mitochondrial isolation buffer composition for use in performing the method of claim 1, said composition comprising:
a buffering agent ;
a chelating agent;
a sugar;
an agent that acts as a membrane stabilizer and/or oxygen radical scavenger and/or binder of Ca²+ and/or binder of free fatty acid; and
a serine protease inhibitor.

20. The mitochondrial isolation buffer composition of claim 19, wherein the composition does not comprise an antibiotic.

21. A mitochondrial storing buffer composition for use in performing the method of claim 1, said composition comprising:
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²+ and/or binder of free fatty acid.

22. The mitochondrial storing buffer composition of claim 21, wherein the composition does not comprise an antibiotic.

23. A kit comprising a donor mitochondria composition in a unit dosage effective to treat traumatic brain injury (TBI) in a subject, said donor mitochondria composition comprising:
mitochondria isolated from tissue of a donor;
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²+ and/or binder of free fatty acid.

24. The kit of claim 23, wherein the donor and the subject are not an HLA (human leukocyte antigen) match.

25. The kit of claim 23, wherein the donor mitochondria composition does not comprise an antibiotic.

26. The kit of claim 23, further comprising instructions for optimizing the dose and/or frequency and/or route of administration of the composition.

27. A method of treating traumatic brain injury (TBI), the method comprising:
administering to a subject a composition comprising allogeneic mitochondria.

28. The method of claim 27, wherein the subject has experienced a TBI.

29. The method of claim 27, wherein the subject is suffering from a TBI symptom.

30. The method of claim 27, wherein the subject has experienced a mild traumatic brain injury (mTBI).

31. The method of claim 27, wherein the subject is suffering from a mTBI symptom.

32. The method of claim 27, wherein the subject is administered the composition at the time of an injury that may result in a TBI.

33. The method of claim 27, wherein the subject is administered the composition after a period of time of a TBI or a suspected TBI.

34. The method of claim 28, wherein the TBI results in a loss of mitochondria in the subject.

35. The method of claim 28, wherein the TBI results in mitochondrial injury dysfunction.

36. The method of claim 27, wherein the method further comprises isolating mitochondria.

37. The method of claim 36, wherein the method comprises isolating mitochondria from a donor other than the subject.

38. The method of claim 36, wherein the method comprises isolating mitochondria from the subject.

39. The method of claim 27, wherein the method further comprises creating a cell bank from a donor other than the subject.

40. The method of claim 39, wherein the cell bank comprises fibroblasts.

41. The method of claim 39, wherein the cell bank comprises mesenchymal stromal cells (MSCs).

42. The method of claim 27, wherein the method comprises obtaining a tissue biopsy from a donor.

43. The method of claim 27, wherein the method comprises creating a tissue bank from a donor.

44. The method of claim 27, wherein the composition further comprises a mitochondrial storing buffer having a potassium ion concentration safe for administration to humans.

45. The method of claim 27, wherein the administering step comprises parenterally administering at least one-unit dose of said composition to the subject.

46. The method of claim 45, wherein the administering step comprises both intramuscular injection and intravenous injection of said composition to said subject.

47. The method of claim 36, wherein isolating the mitochondria comprises preparing cell lysate from tissue via tissue dissociation.

48. The method of claim 36, wherein isolating the mitochondria comprises using a mitochondrial isolation buffer comprising a serine protease inhibitor.

49. The method of claim 36, comprising isolating the mitochondria without using an antibiotic.

50. The method of claims 27, wherein the subject and the donor from which mitochondria are obtained are not an HLA (human leukocyte antigen) match.

51. The method of claim 27, wherein the composition administered to the subject does not comprise an antibiotic.

52. The method of claim 36, wherein the isolating step is conducted using a mitochondrial isolation buffer composition.

53. The method of claim 52, wherein the mitochondrial isolation buffer composition comprises:
a buffering agent;
a chelating agent;
a sugar;
an agent that acts as a membrane stabilizer and/or oxygen radical scavenger and/or binder of Ca²+ and/or binder of free fatty acid; and
a serine protease inhibitor.

54. The mitochondrial isolation buffer composition of claim 53, wherein the composition does not comprise an antibiotic.

55. The method of claim 36, further comprising storing the isolated mitochondria at a temperature below −40° C.

56. The method of claim 27, comprising administering to the subject an iron-chelating agent.

57. The method of claim 27, comprising administering to the subject an antioxidant and/or a probiotic.

58. A mitochondrial isolation buffer composition for use in performing the method of claim 27, said composition comprising:
a buffering agent;
a chelating agent;
a sugar;
an agent that acts as a membrane stabilizer and/or oxygen radical scavenger and/or binder of Ca²+ and/or binder of free fatty acid; and
a serine protease inhibitor.

59. The mitochondrial isolation buffer composition of claim 58, wherein the composition does not comprise an antibiotic.

60. A mitochondrial storing buffer composition for use in performing the method of claim 27, said composition comprising:
one or more buffering agents;
a source of magnesium ion;
a chelating agent;
a sugar;
an antioxidant;
a cytoprotective agent that binds to calcium ion; and
an agent that acts as a membrane stabilizer and/or oxygen radical scavenger and/or binder of Ca²+ and/or binder of free fatty acid.

61. The mitochondrial storing buffer composition of claim 60, wherein the composition does not comprise an antibiotic.

62. Use of allogeneic mitochondria for the treatment of traumatic brain injury (TBI).

63. Use of allogeneic mitochondria for the treatment of traumatic brain injury (TBI), wherein the allogeneic mitochondria is administered to a subject suffering from traumatic brain injury (TBI)-associated mitochondrial damage.

64. The use of claim 63, wherein the subject has experienced a TBI

65. The use of claim 63, wherein the subject is suffering from a TBI symptom.

66. The use of claim 63, wherein the subject has experienced a mild traumatic brain injury (mTBI).

67. The use of claim 63, wherein the subject is suffering from a mTBI symptom.

68. The use of claim 63, wherein the use comprises administering a composition comprising the allogeneic mitochondria the time of an injury that may result in a TBI.

69. The use of claim 63, wherein the use comprises administering a composition comprising the allogeneic mitochondria after a period of time of a TBI or a suspected TBI.

70. The use of claim 63, wherein the subject is suffering from a loss of mitochondria.

71. The use of claim 63, wherein the subject is suffering from mitochondrial injury dysfunction.
